(19) United States
(12) Patent Application Publication
(10) Pub. No.: US 2019/0076047 A1
(43) Pub. Date: Mar. 14, 2019

(54) ELECTROMYOGRAPHY-ENHANCED BODY AREA NETWORK SYSTEM AND METHOD

(71) Applicant: Sony Mobile Communications Inc., Tokyo (JP)

(72) Inventors: Ola Thörn, Limhamn (SE); Magnus Midholt, Lund (SE); Kåre Agardh, Lund (SE); Erik Bengtsson, Lund (SE)

(21) Appl. No.: 16/076,619

(22) PCT Filed: Feb. 9, 2016
(86) PCT No.: PCT/US2016/017117

§ 371 Date: Aug. 8, 2018

(51) Int. Cl. A61B 5/0488; G06F 3/01

(52) U.S. Cl. CPC A61B 5/0488; G06F 3/017; G06F 3/015

(57) ABSTRACT

Determining the location of a body area network enabled device on a user's body and using the location information to determine whether to send instructions or data to the device over the body area network, includes integrating an EMG sensor into a device that is wearable or otherwise meant to be in contact with or close proximity to an intended area of a user's body. The integrated EMG sensor may detect the electrical potential of the user's body at the point of contact. Variance of electrical potential across the user's body may be used to determine information regarding the location of the sensor, and thus, the location of the device on the user's body. The location of the device, in turn, may be used to determine whether to send instructions or data to the device over the body area network.

### TECHNICAL FIELD

The present disclosure relates generally to electronic devices and, more particularly, to electronic devices configured to communicate with each other through the user's own body as a communication medium.

### BACKGROUND ART

There are many devices available to consumers that are designed to be used as companion, or secondary, products to primary smartphone devices. Many such devices may be configured as wearable devices, such as a ring, a necklace, a watch, eyeglasses, a bracelet, a wristband, or a headset with speakers and a microphone.

A commonality among these devices is that they need an interface and a transmission medium in order to transmit data between the primary device and the companion, or secondary devices. Body-coupled communication (BCC) is an emerging technology in which the human body serves as a transmission medium between the devices. For example, a communication signal may travel on, proximate to, or through the human body. A transmitter at one device and a receiver at the other device are used to transmit a body-coupled signal and receive the body-coupled signal.

While there are a number of properties related to body-coupled communication as compared to other forms of communication (e.g., wired transmission, or more traditional “over-the-air” wireless transmission), such as power usage, security, resource utilization, etc., there is still room for improvement in the seamlessness of the user's experience with devices using body-coupled communication over a body area network (BAN).

One issue that currently arises with the use of secondary BAN enabled devices is the lack of an ability to discern whether the device is situated or worn as intended, or merely in contact, intentionally or unintentionally, with a different part of the user's body. This issue can lead to unintended instructions being carried out on the secondary device.

### SUMMARY

According to one aspect of the disclosure, disclosed is a body area network system. The system includes a first body area network (BAN) enabled device comprising a control circuit that operatively controls the first BAN enabled device; a BAN communication interface; and an electromyography (EMG) sensor configured to sense an electrical potential of an area of tissue in contact with the EMG sensor; a body area detection engine configured to receive a first signal communicated from the EMG sensor, wherein the first signal indicates the sensed electrical potential of an area of tissue in contact with the EMG sensor; evaluate the indicated electrical potential with respect to a predetermined value; determine, based on the evaluation, that the first device is operatively positioned with respect to a user; and cause the BAN communication interface to at least one of communicate a notification to a second BAN enabled device, the notification notifying the second device that the first device is operatively positioned; or receive one or more functional instructions from the second BAN enabled device.

According to one embodiment of the system, the body area detection engine is part of the control circuit.

According to one embodiment of the system, the body area detection engine is part of a control circuit of the second BAN enabled device.

According to one embodiment of the system, the body area detection engine is housed partially in the housing of the first BAN enabled device and partially in the housing of the second BAN enabled device.

According to one embodiment of the system, the predetermined value is stored on the first BAN enabled device.

According to one embodiment of the system, the predetermined value is stored on the second BAN enabled device.

According to one embodiment of the system, the evaluation comprises comparing the indicated electrical potential and the predetermined value; and the evaluation indicates that the secondary BAN enabled device is operatively positioned on a user when the indicated electrical potential and the stored value match.

According to one embodiment of the system, the stored value represents a range of values, and the indicated electrical potential matches the stored value when the indicated electrical potential matches any value in the range of values.

According to one embodiment of the system, the EMG sensor communicates a second signal indicating a second sensed electrical potential of a second area of tissue contacted by the EMG sensor; the body area detection engine evaluates the other indicated electrical potential with respect to a second predetermined value; and the evaluation comprises determining, based on the electrical potential indicated by the second signal and the second predetermined value, that a predetermined gesture has been performed by the user.

According to one embodiment of the system, the second signal is communicated by an other EMG sensor.

According to one embodiment of the system, the second signal is communicated by a third BAN enabled device.

According to one embodiment of the system, the first device contains an other input sensor and the evaluation comprises further determining that a gesture has been performed by the user based on input from the other input sensor.

According to one embodiment of the system, the functional instructions are user defined functional instructions.

According to one embodiment of the system, the predetermined value is recorded through a user-performed learning procedure.

According to one aspect of the disclosure, disclosed is a method for determining that a first BAN enabled device is operatively positioned comprising sensing, via an EMG sensor integrated into the first BAN enabled device, an electrical potential of an area of tissue in contact with the EMG sensor; communicating a first signal from the EMG sensor to a body area detection engine, wherein the signal indicates the sensed electrical potential of the tissue in contact with the EMG sensor; evaluating, by the body area detection engine, the indicated electrical potential with respect to a predetermined value; determining, based on the evaluating, that the first device is operatively positioned with respect to a user; and causing a BAN communication interface integrated into the first BAN enabled device to at least one of communicate a notification to a second BAN enabled device, the notification notifying the second device that the first device is operatively positioned; or receive one or more functional instructions from the second BAN enabled device.

According to one embodiment of the method, the body area detection engine is hosted by the first BAN enabled device.

According to one embodiment of the method, the body area detection engine is hosted by the second BAN enabled device.

According to one embodiment of the method, the body area detection engine is hosted partially by the first BAN enabled device and hosted partially by the second BAN enabled device.

According to one embodiment of the method, the predetermined value is stored on the first BAN enabled device.

According to one embodiment of the method, the predetermined value is stored on the second BAN enabled device.

According to one embodiment of the method, the evaluating includes comparing the indicated electrical potential and the predetermined value; and the evaluating further includes indicating that the secondary BAN enabled device is operatively positioned on a user when the indicated electrical potential and the predetermined value match.

According to one embodiment of the method, the predetermined value represents a range of values, and the indicated electrical potential matches the stored value when the indicated electrical potential matches any value in the range of values.

According to one embodiment of the method, the method further includes communicating a second signal indicating a second sensed electrical potential of a second area of tissue contacted by the EMG sensor to the body area detection module; evaluating the second indicated electrical potential with respect to a second predetermined value; and determining, based on the electrical potential indicated by the second signal and the second predetermined value, that a predetermined gesture has been performed by the user.

According to one embodiment of the method, the second signal is communicated by an other EMG sensor.

According to one embodiment of the method, the second signal is communicated by a third BAN enabled device.

According to one embodiment of the method, the first device contains an other input sensor and the evaluation comprises further determining that a gesture has been performed by the user based on input from the other input sensor.

According to one embodiment of the method, the functional instructions are user defined functional instructions.

According to one embodiment of the method, the predetermined value is recorded through a user-performed learning procedure.

According to one embodiment of the method, the second predetermined value is recorded through a user-performed learning procedure.

### BRIEF DESCRIPTION OF THE DRAWINGS

FIGS. 1A-1G are schematic diagrams of exemplary BAN enabled devices.

FIG. 2 is a schematic block diagram of an exemplary primary BAN enabled device.

FIG. 3 is a schematic block diagram of an exemplary secondary BAN enabled device.

FIG. 4 is a schematic block diagram of an exemplary body area detection engine.

FIG. 5 is a flow-diagram of exemplary operative position detection logic.

FIG. 6 is a flow-diagram of exemplary body area detection logic.

### DETAILED DESCRIPTION

Embodiments will now be described with reference to the drawings, wherein like reference numerals are used to refer to like elements throughout. It will be understood that the figures are not necessarily to scale. Features that are described and/or illustrated with respect to one embodiment may be used in the same way or in a similar way in one or more other embodiments and/or in combination with or instead of the features of the other embodiments.

Described below in conjunction with the appended figures are various embodiments of systems and methods for using electromyography (EMG) and EMG sensors in conjunction with devices configured to communicate over a body area network (BAN) using body-coupled communication. The disclosed techniques are primarily described in the context of smartphones or other primary personal electronic devices that communicate with secondary personal electronic devices. However, the techniques may be applied in other contexts such as personal devices communicating via body-coupled communication with home electronic devices, public devices, or public information systems.

The techniques involve integrating an EMG sensor into electronic devices that are wearable or otherwise meant to be in contact with or close proximity to an intended area of the user's body. The integrated EMG sensor may detect the electrical potential of the user's body at the point of contact. Because the electrical potential of the human body varies across different parts of the body, this variance may be used to determine information regarding the location of the sensor, and thus, the location of the secondary device on the user's body. The location of the secondary device, in turn, may be used to determine whether to send certain instructions or data to the secondary device.

As used herein, the term “primary device,” “primary communication device,” or “primary BAN enabled communication device” includes any primary communication device that includes body coupled communication (BCC) capabilities. Exemplary primary devices include mobile phones, smartphones, laptops (such as standard, ultra-portables, netbooks, Chromebook®, and micro laptops), handheld computers, portable digital assistants, tablet computers, touch pads, or gaming devices. The term “secondary device,” or “secondary BAN enabled device” will generally refer to accessories to mobile phones or other primary devices that are intended to work in conjunction with primary devices (e.g., wearable communication devices in the form of headphones, headsets, visors, goggles, bracelets, wristbands, necklaces, watches, headbands, rings, etc.). However, these lists are for descriptive purposes, are not exhaustive, and it is contemplated that BAN enabled devices may overlap widely in actual functionality.

Body Area Network (BAN) standards such as IEEE's 802.15.6 or Sony's CCCC are enabling new possibilities for commercialized BAN devices. The first BAN enabled devices were developed for use in the medical field, since Body Coupled Communications (BCC) is a communication standard optimized for low power devices and operation on, in or around the human body.

BCC, also referred to as Body-Based Communication (BBC) or Near-Body Communication (NBC), has been developed as an alternative or supplement to short range radio frequency (RF) communication as a basis for Personal Area Network (PAN) communication. BCC allows for an exchange of information between a plurality of devices that are in contact with, or in very close proximity to, a body, or in some instances, multiple bodies. This may be achieved by the transmitting BCC/BAN contact (also referred to as an electrode or an antenna) that provides a capacitive or galvanic coupling of low-energy electrical fields onto the body surface (i.e., leakage currents), with a small magnitude set to spread out over the human body. The small currents are then detected by a receiver BCC contact located on the same body. Thus, signals are conveyed over the body instead of through the air. As such, the communication is confined to the volume of space close to the body in contrast to RF communications, where a much larger volume of space is covered. Therefore, communication is possible between devices situated on, connected to, or placed close to the body. As an additional advantage, power consumption of BCC-antennas is very low.

Electromyography (EMG) is a technique for evaluating and recording the electrical potential (or voltage) of living tissue—primarily the tissue of a human body. EMG technology also originated in the medical field, and has a variety of clinical and biomedical applications. The electrical potential of the tissue in the human body is caused by an imbalance of ions between the two sides of a cell membrane. There are at least two known types of electrical potentials that can be detected in the human body: resting potential and action potential. Resting potential is the relatively static electrical potential of membrane cells. Action potential is the specific dynamic electrochemical phenomena that occurs in excitable cells such as neurons, muscles, and some secretory cells in glands. Action potential occurs when a muscle is flexed, tensed, or otherwise exercised. Resting potential is always present and can be measured in almost all types of cells of the human body.

Resting potential has two properties that make it useful for identifying a specific area of the human body. First, the resting potential is different at each part of the body. Second, the resting potential is relatively stable over time and against stimulation, because it is determined by the cells' static properties. Thus, it can be expected that at a given part of the body there will be a predictable electrical potential. For example, the resting membrane potential for skeletal muscle cells is approximately −95 mV, and for smooth muscle cells is approximately −60 mV. Neurons have a resting potential of −60 to −70 mV. Additionally, each area across the surface of the skin also has a unique resting potential. For example, the lower arm will have a different resting potential than the upper arm, and the palm will have a different resting potential than the wrist, etc. This resting potential can be sensed and used to determine a body area location of the sensor.

Consistent with embodiments described herein, a primary BAN enabled communication device and/or a secondary BAN enabled device may include a body area detection engine, including body area detection logic, for determining what area of the user's body is in contact with the secondary BAN enabled device. This information may be used to determine when to execute certain communications or procedures, referred to herein as “functional instructions.” For instance, and as will be described in more detail below, it may be desirable to execute functional instructions only when it is determined that a secondary BAN enabled device is positioned or worn in its intended location on the user's body.

As described in detail below, body area detection logic may make a determination of the area of a user's body in contact with a secondary BAN enabled device based on signals received from an EMG sensor embedded in the secondary BAN enabled device. In one embodiment, the signals from the EMG sensor may be communicated between the primary BAN enabled communication device and the secondary BAN enabled device via BAN contacts on the respective devices that are configured to communicate over the user's body (i.e., the signals may be communicated via a BAN). In this scenario, the signals from the EMG sensor are evaluated, and a determination is made, on the primary BAN enabled device. In another embodiment, signals from the EMG sensor are evaluated on, and the determination is made on the secondary BAN enabled device.

If body area detection logic determines that the secondary BAN enabled device is positioned or worn by the user in its intended location, then functional instructions may be requested by the secondary device, and/or sent by the primary device to the secondary device. By only executing functional instructions (i.e., instructions intended to execute only when a secondary BAN enabled device is positioned or worn in its intended location) after EMG signals are communicated and evaluated to determine that the device is worn as intended, the devices can eliminate such instructions being erroneously sent merely because the secondary device is in random contact with the user's body. For example, if a BAN enabled headset is merely picked up and held in a user's hand, audio functions will not be carried out using the disclosed approach. But when the user places the headset at his or her ear, audio functions may be carried out.

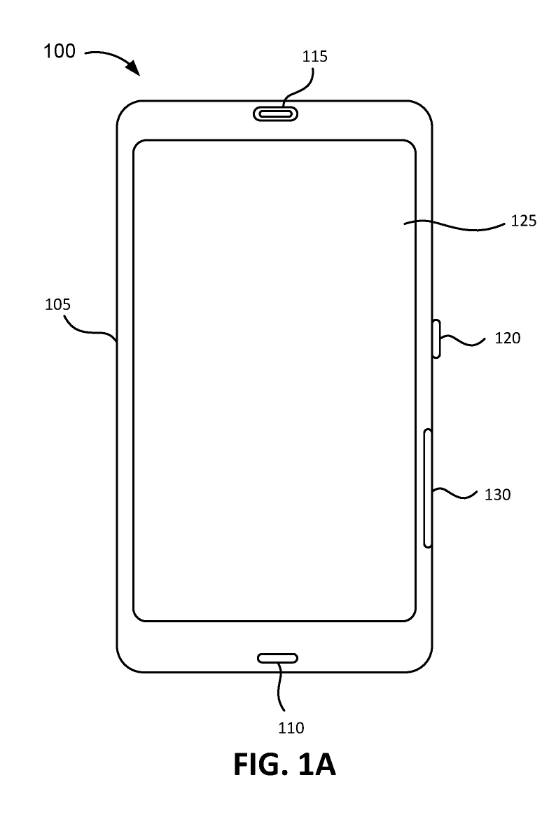

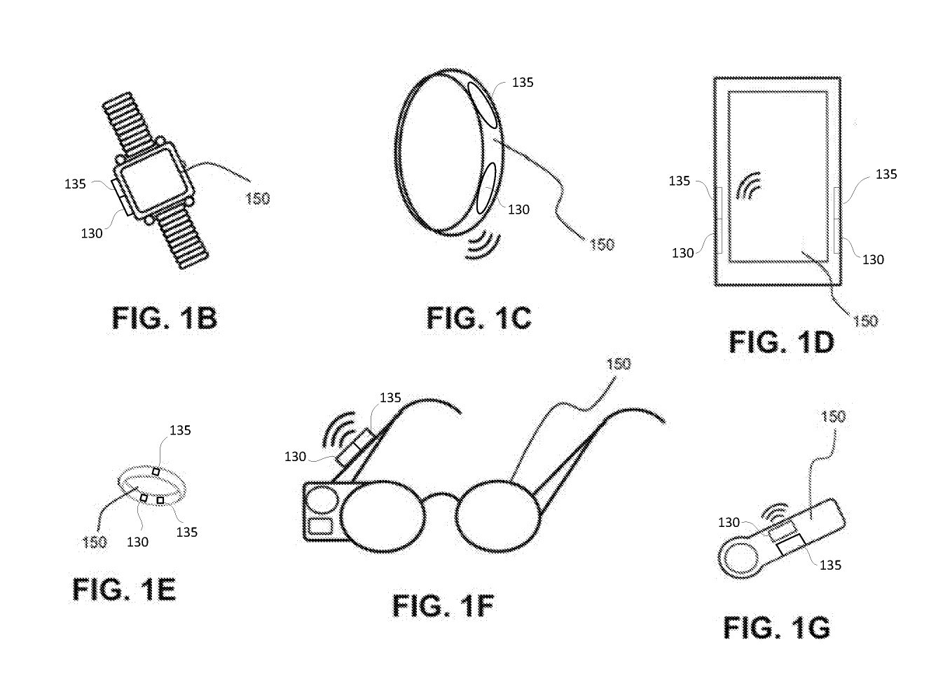

FIG. 1A is a diagram of an exemplary primary BAN enabled communication device 100 (also referred to simply as “communication device 100,” “primary device 100,” or “device 100”) consistent with embodiments described herein. As described herein, communication device 100 may be generally referred to below as a mobile phone or smartphone, although, as briefly described above, device 100 may include any device that is capable of BAN communications.

As shown in FIG. 1A, communication device 100 comprises a housing 105, a microphone 110, a speaker 115, a button 120, a display 125, and at least one BAN contact 130.

According to other embodiments, communication device 100 may comprise fewer components, additional components, different components, or a different arrangement of components than those illustrated in FIG. 1A and described herein. For example, communication device 100 may include a port (e.g., a headphone port, a Universal Serial Bus (USB) port, a High Definition Multimedia Interface (HDMI) port, or some other type of input port and/or output port, etc.), a camera, a keypad, a keyboard, a biometric reader (e.g., a retina scanner, etc.), etc. Additionally, or alternatively, communication device 100 may take the form of a different configuration (e.g., a slider, a clamshell, a swivel, etc.) than the configuration illustrated in FIG. 1A. Also, according to some embodiments, BAN contact 130 may comprise a plurality of regions or may comprise an entirety of housing 105.

Housing 105 comprises a structure to contain components of communication device 100. For example, housing 105 may be formed from plastic, metal, or some other type of material. Housing 105 may support microphone 110, speaker 115, button 120, display 125, and BAN contact 130.

Microphone 110 is capable of transducing a sound wave to a corresponding electrical signal. For example, a user may speak into microphone 110 during a telephone call or to execute a voice command. Speaker 115 is capable of transducing an electrical signal to a corresponding sound wave. For example, the user may listen to music or to a calling party through speaker 115.

Button 120 provides an input to communication device 100. Button 120 may provide a single or dedicated function (e.g., power) or multiple functions. For example, button 120 may enable deactivation of display 125 as well as the complete powering on and off of communication device 100. Alternatively, button 120 may provide performing a camera function, volume control, etc. Button 120 may be a hardware button. For example, button 120 may be a button, a rocker style button, etc. Additionally, or alternatively, button 120 may be a capacitive-touch button.

Display 125 operates as an output component. For example, display 125 may comprise a liquid crystal display (LCD), a plasma display panel (PDP), a field emission display (FED), a thin film transistor (TFT) display, or some other type of display technology (e.g., organic LED (OLED), active matrix OLED (AMOLED), etc.). Display 125 is capable of displaying text, pictures, video, images (e.g., icons, objects, etc.). Display 125 may also be capable of providing haptic or tactile feedback.

Additionally, display 125 may operate as an input component. For example, display 125 may comprise a touch-sensitive screen. Display 125 may be implemented using a variety of sensing technologies, such as capacitive sensing, surface acoustic wave sensing, resistive sensing, optical sensing, pressure sensing, infrared sensing, or gesture sensing. In such instances, display 125 may operate as a single-point input device (e.g., capable of sensing a single touch) or a multipoint input device (e.g., capable of sensing multiple touches that occur at the same time). Additionally, or alternatively, display 125 may comprise a touchless screen (e.g., having air-touch, or air-gesture capabilities).

Referring now to FIGS. 1B-1G, illustrated are diagrams of exemplary secondary BAN enabled devices 150 (also referred to as “secondary device 150,” and “device 150”). In general, a secondary device 150 refers to a wireless communication device configured to be worn or touched by a person during use and which is further configured to communicate with primary BAN enabled communication device 100 via a BAN. Examples of such BAN enabled devices include a watch, as shown in FIG. 1B, a bracelet, as shown in FIG. 10, a smartphone or tablet, as shown in FIG. 1D, a ring, as shown in FIG. 1E, a pair of eyeglasses, as shown in FIG. 1F, and a headset or earpiece, as shown in FIG. 1G. It should be understood that the illustrated examples are not exhaustive, and any suitable BAN enabled device may be implemented in accordance with embodiments described herein, and include alternatives, such as skin contact patches, headphones, necklaces, clothing, 3D visors, helmets, etc. Further included alternatives not shown in the figures are BAN enabled household or publicly accessible items such as BAN enabled door handles/knobs/locks, payment stations, turnstiles, electronic calendars, elevator control panels, etc.

With reference to FIGS. 1A-1G, communication device 100, and each secondary BAN device 150 include at least one BAN contact 130. BAN contact 130 may include a conductive portion integrated within the housing of the device 100/150, and may be coupled internally to a BAN antenna, which in turn may be coupled to a BAN transceiver, described below. Alternatively, BAN antenna may be integrated with BAN contact 130. As shown in the corresponding figures, BAN contact 130 may be provided in a region of communication device 100 and/or secondary BAN device 150 that is typically adjacent an intended portion of a user during normal use. Further, as described above, BAN contact 130 may include multiple regions for further ensuring that at least one BAN contact 130 is in contact with the user, when the user uses or wears the device.

Consistent with embodiments described herein, BAN contact 130 may include or communicate with a body sensor and a BAN electrode to determine on-body contact and transmit signals to, and receive signals from, the body. In operation, the body sensor may enable a determination that communication device 100 is in contact with a human body, and the BAN electrode may form the medium through which BAN signals are output to and received from the user's body.

In accordance with embodiments described herein, each secondary BAN device 150 also includes at least one EMG sensor 135. EMG sensor 135 may include a sensing portion integrated within the housing of secondary BAN device 150. EMG sensor 135 may be provided in a region of secondary BAN device 150 that typically contacts a user during use of device 150. Further, EMG sensor 135 may include multiple regions for further ensuring that at least one EMG sensor 135 is in contact with the user when the user possesses or wears secondary device 150. In one embodiment, EMG sensor shares an electrode with BAN contact 130. In still another embodiment, EMG sensor comprises the entirety of the housing of secondary device 150.

In accordance with embodiments described herein, EMG sensor 135 may contain at least one electrode. Embodiments of EMG sensor 135 may include monopolar arrangements (i.e., a single electrode and a ground), bipolar arrangements (i.e., two electrodes and a ground), or any suitable arrangement of electrodes and/or grounds. Further, EMG sensor 135 may include active surface electrodes (i.e., those having built-in amplifiers at the electrode site), passive surface electrodes, or any other suitable type or arrangement of electrodes.

In accordance with an embodiment, EMG sensor 135 may be merely an EMG sensor of the type and/or arrangements described above, or it may incorporate other types of sensors and/or electrodes, such as capacitive sensors and copper electrodes, used to gather additional information about the area of the body with which EMG sensor 135 is in contact, or the proximity of EMG sensor 135 to the user's body.

Consistent with embodiments described herein, EMG sensor 135 may include, or communicate with, an analog to digital converter (not shown). The converter may receive analog signals from the EMG sensor 135 corresponding to the electrical potential of the tissue in contact with EMG sensor 135 and convert the analog signals into digital signals or values for use by the control circuit, memory and software of secondary BAN device 150 and/or primary BAN device 100.

As used herein in the context of either BAN contact 130 or EMG sensor 135, the phrase “in contact with” means in direct contact with, or in close enough proximity to an area of a user's body to allow either BAN contact 130 or EMG sensor 135 to function as intended. “In contact” may include the presence of an intervening material, such as air, water, clothing, hair, housing material, or protective coating, etc.

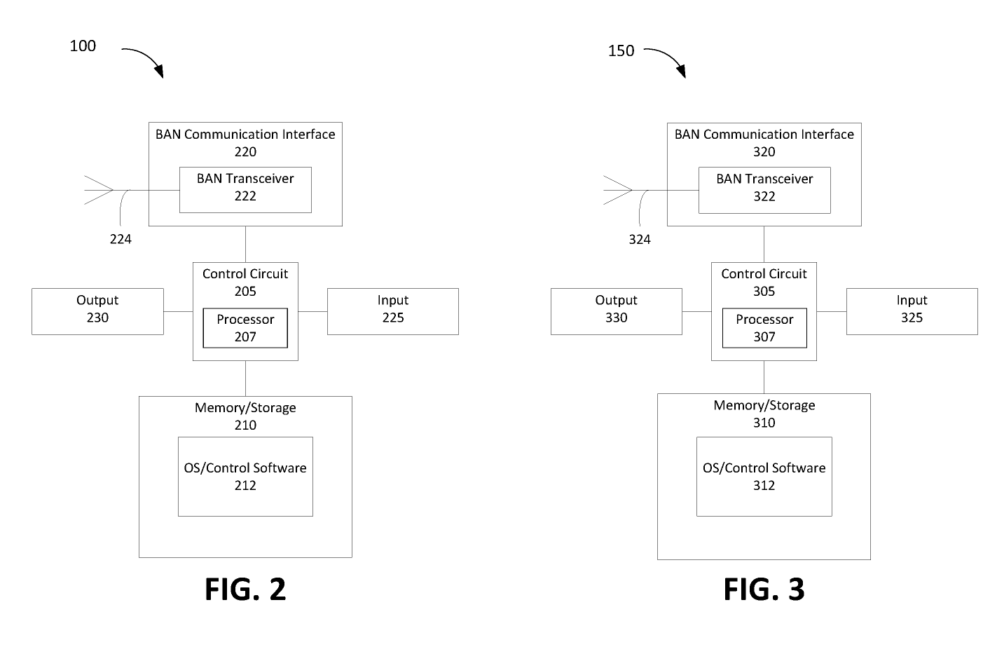

FIG. 2 is a diagram illustrating exemplary components of communication device 100. As illustrated, communication device 100 includes a control circuit 205, a memory/storage 210, operating system (OS)/Control software 212, a communication interface 220, an input 225, and an output 230. Control circuit 205 is responsible for the overall operation of device 100. Control circuit 205 may be implemented as, or include, hardware (e.g., a microprocessor, microcontroller, central processing unit (CPU), etc.) or a combination of hardware and software (e.g., a system-on-chip (SoC), an application-specific integrated circuit (ASIC), etc.). In one embodiment, control circuit 205 includes a processor 207 that executes operating instructions. The processor 207 of control circuit 205 may execute code in order to carry out the operation of device 100. According to other embodiments, communication device 100 may include fewer components, additional components, different components, and/or a different arrangement of components than those illustrated in FIG. 2 and described herein.

Likewise, FIG. 3 is a diagram illustrating exemplary components of secondary BAN device 150. As illustrated, secondary BAN device 150 includes a control circuit 305, a memory/storage 310, OS/control software 312, a communication interface 320, an input 325, and an output 330. Control circuit 305 is responsible for the overall operation of device 150. Control circuit 305 may be implemented as, or include, hardware (e.g., a microprocessor, microcontroller, central processing unit (CPU), etc.) or a combination of hardware and software (e.g., a system-on-chip (SoC), an application-specific integrated circuit (ASIC), etc.). In one embodiment, control circuit 305 includes a processor 307 that executes operating instructions. The processor 307 of control circuit 305 may execute code in order to carry out the operation of device 150. According to other embodiments, secondary BAN enabled device 150 may include fewer components, additional components, different components, and/or a different arrangement of components than those illustrated in FIG. 3 and described herein.

With continued reference to FIGS. 2-3, memory/storage 210/310 includes one or multiple memories and/or one or multiple other types of storage mediums. For example, memory/storage 210/310 may include a random access memory (RAM), a dynamic random access memory (DRAM), a cache, a read only memory (ROM), a programmable read only memory (PROM), flash memory, and/or some other type of memory. Memory/storage 210/310 may include a hard disk (e.g., a magnetic disk, an optical disk, a magneto-optic disk, a solid state disk, etc.). In a typical arrangement, the memory/storage 210/310 includes a non-volatile memory for long term data storage and a volatile memory that functions as the system memory for control circuit 205/305. The memory/storage 210/310 may exchange data with control circuit 205/305 over a data bus. Accompanying control lines, and an address bus between memory/storage 210/310 and control circuit 205/305, respectively, may also be present. Memory/storage 210/310 is considered a non-transitory computer readable medium.

OS/control software 212/312 may include device firmware, an operating system (OS), or a variety of applications that may execute on the devices 100/150. By way of example, depending on the implementation of device 100/150, the operating system may correspond to iOS, Android, Windows Phone, Symbian, or another type of operating system (e.g., proprietary, BlackBerry OS, Windows, Tizen, Linux, Unix, etc.). Additionally software 212/312 may comprise a telephone application, a multi-media application, an e-mail application, a contacts application, a calendar application, an instant messaging application, a web browsing application, a location-based application (e.g., a Global Positioning System (GPS)-based application, etc.), a camera application, media player application, etc. According to embodiments described herein, OS/control software 212/312 includes one or more applications configured to support the exchange of information between communication device 100 and secondary BAN device 150.

With continued reference to FIGS. 2-3, communication interface 220/320 permits device 100/150 to communicate with other devices, networks, systems, etc. Communication interface 220/320 may include one or multiple wireless interfaces and/or wired interfaces. Communication interface 220/320 may include one or multiple transmitters, receivers, and/or transceivers. Communication interface 220/320 operates according to one or multiple protocols, communication standards, and/or the like. In particular, as described above, communication interface 220/320 includes at least a BAN transceiver (or a discrete receiver and transmitter) 222/322 and BAN antenna 224/324 for interfacing with BAN contact 130 (i.e., the BAN electrode) to transmit and receive BAN signals from other BAN enabled devices, such as between primary BAN device 100 and secondary BAN device 150. In some embodiments, communication interface 220/320 includes other transmitters and transceivers to enable communication via other protocols, such as Bluetooth®, near field communication (NFC), Wi-Fi, as well as long range wireless communications, such as 3G, LTE (long term evolution), etc.

Output 230/330 permits an output from device 100/150. For example, output 230/330 may include a speaker, a display, a light, an output port, a vibration device, and/or some other type of output component. In the case of secondary BAN device 150, in some instances, device 150 may not include an output 330, such as when device 150 comprises a ring.

Input 225/325 permits an input into device 100/150. For example, input 225/325 may include a button, a switch, a touch pad, an input port, speech recognition logic, and/or a display (e.g., a touch display, a touchless display). Input 225/325 may include sensors such as accelerometers, gyroscopes, etc. As described above, and according to an embodiment, input 225/325 includes at least one body sensor in BAN contact 130. Further, input 325 includes at least one EMG sensor 135.

When using a secondary BAN enabled device 150 in conjunction with a primary BAN enabled communication device 100, the respective devices may initiate communication with each other when each device's respective BAN contact 130 is in contact with a user's body. As discussed above in relation to input 225/325 of FIGS. 2 and 3, respectively, BAN contact 130 may include a sensor configured to detect when the device 100/150 is in contact with a user's body. For example, such a sensor may be configured to detect body temperature, blood flow, pulse, etc. In other embodiments, the body sensor in BAN contact 130 may include an accelerometer, an optical sensor, etc. In still other embodiments, the body sensor of BAN contact 130 includes a capacitive touch system. Such a capacitive touch system may have low power consumption (e.g., less than 30 Micro Amp), and a sensitivity range of 20-30 mm, such that the device may be may be loosely coupled around a body part, such as an arm, and still give a signal. In still another embodiment, BAN contact 130 may include a combination of a capacitive touch system, an accelerometer, etc. When BAN contact 130 is initially placed into contact with the user's body, the capacitive touch system, or other sensor, may notify control circuit 205/305 that the device is on a body.

Notwithstanding the manner in which an on-body contact is determined, signals coming from the body sensor of BAN contact 130 may be received at the control circuit 205/305 and used as a trigger to initiate the BAN interface to attempt to establish a BAN link with other body coupled devices. For example, when a user puts a pair of BAN enabled eye glasses 150 on, BAN contact 130 may sense that the glasses are in contact with the user and communicate one or more signals to control circuit 305. Upon receiving the signals from BAN contact 130, control circuit 305 may be configured to attempt to establish a BAN link with primary device 100. Conversely, if a user is already wearing BAN enabled glasses 150, and the user then puts primary device 100 in his or her pocket, thus causing BAN contact 130 of primary device 100 to sense on-body contact, BAN contact 130 of primary device 100 may communicate one or more signals to control circuit 205. Upon receiving the signals from BAN contact 130, control circuit 205 may be configured to attempt to establish a BAN link with secondary device 150.

Generally, once BAN communication is established as described above, primary device 100 and secondary device 150 may communicate data and/or instructions to each other over the established BAN. Typically, these data and/or instructions facilitate the carrying out of some functionality associated with secondary device 150. For example, when secondary device 150 is a BAN enabled headset with speakers, primary device 100 may communicate data in the form of digital music to headset 150. When secondary device 150 is a pair of BAN enabled glasses with an integrated camera, primary device 100 may communicate instructions to start the camera recording, and glasses 150 may communicated data in the form of the digital recording back to primary device 100 for storage in memory/storage 210. Moreover, when secondary device 150 is a door handle with a latch, primary device 100 may communicate instructions to cycle the latch, thereby allowing the door to open, etc. Data and/or instructions beyond the mere facilitation of BAN communications between devices 100 and 150, and that are related to the intended and/or central functionality of any secondary device 150, such as those examples given above, are referred to herein as “functionality instructions.” It is typically desirable to send functionality instructions only when secondary device 150 is operatively positioned on the user's body.

In accordance with embodiments described herein, a user of a secondary BAN device 150 may possess or wear secondary BAN device 150 such that secondary BAN device 150 is positioned on the user's body in accordance with secondary BAN device's intended purpose, and in a manner that allows secondary BAN device to best carry out its intended and/or central functionality—i.e., secondary BAN device is “operatively positioned” on or with the user. For example, in the case where secondary BAN device 150 is a watch, secondary BAN device 150 is operatively positioned when the user wears secondary BAN device on his or her wrist. When secondary BAN device is a bracelet, secondary BAN device 150 is similarly operatively positioned on the user's wrist. Alternatively, if secondary BAN device 150 is a ring, then secondary BAN device 150 is operatively positioned on the user's finger. If secondary BAN device is a set of headphones, then secondary BAN device is operatively positioned when the headphones are on the user's head and covering one or both ears, etc.

In accordance with an embodiment, secondary BAN device 150 does not need to be worn in order to be considered operatively positioned. For instance, if secondary BAN device 150 is a tablet, or other hand-held device, then secondary BAN device 150 may be considered operatively positioned when the user holds secondary BAN device 150 in his or her hand or hands. If secondary BAN device 150 is a BAN enabled door handle, then secondary device 150 may be considered operatively positioned when a use closes his or her palm around the door handle.

As noted above, it is typically only desirable to communicate/execute functionality instructions once secondary device 150 is operatively positioned on or with the user. This point is illustrated in the scenario where a user picks up a pair of BAN enabled glasses 150 touching BAN contact 130 as he or she holds glasses 150. In this scenario, glasses 150 and primary device 100 may establish BAN communication, and primary device 100 may then send functionality instructions to glasses 150 which instruct a camera integrated into glasses 150 to start recording. Until the glasses 150 are positioned on the face/head as intended, the result is that the video recorded by glasses 150 is likely not a video desired by the user, because the user was not wearing the glasses as they were intended to be worn (i.e., the glasses were not operatively positioned), and the user may have not even known that the glasses were recording while he or she was holding them.

Another example illustrating this point is where secondary BAN enabled device 150 is a door handle with a latch (not shown in the figures). In this scenario, the entirety of the handle may act as BAN contact 130. Further, the door handle may be considered operatively positioned when the door handle is grasped in the palm of a user's hand. The functionality instructions may be to cycle the latch, thereby allowing the door to open. However, should a user inadvertently touch the handle with his or her elbow or arm, BAN communication may be established, and functionality instructions may be communicated from the user's primary device to the BAN enabled handle/latch, thereby cycling the latch when the user had not intended to cycle the latch.

In accordance with embodiments described herein, signals from EMG sensor 135 may be evaluated to determine if secondary BAN device 150 is operatively positioned on or with a user. For example, in one embodiment, operative position detection logic may evaluate a signal communicated from EMG sensor 135 to determine if a given secondary BAN enabled device 150 is operatively positioned.

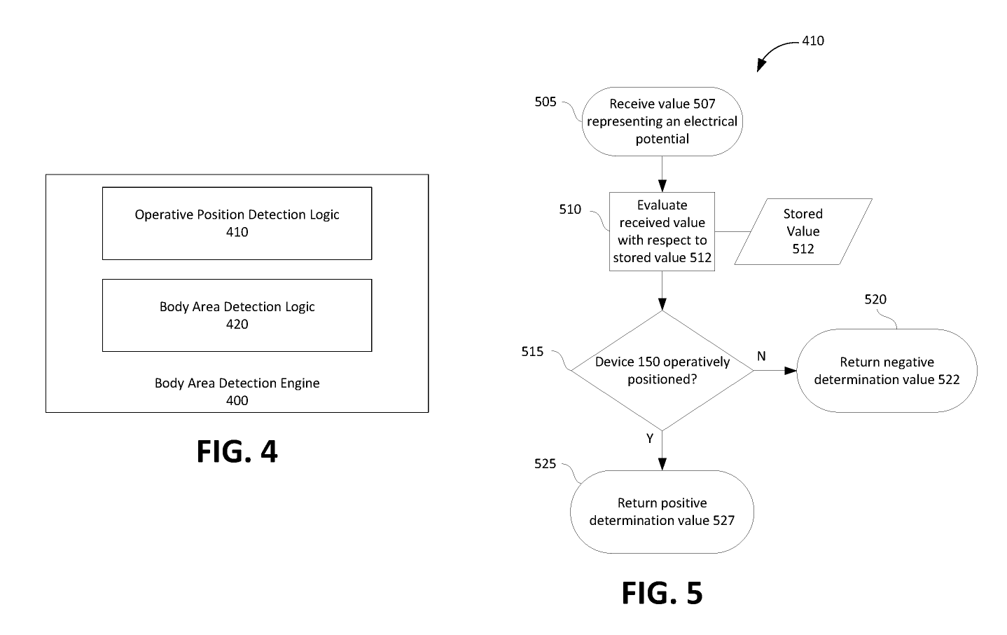

FIG. 4 is a diagram illustrating body area detection engine 400. In accordance with an embodiment, body area detection engine 400 includes operative position detection logic 410 and body area detection logic 420. In accordance with an embodiment, body area detection engine 400 may be configured to receive a signal communicated from EMG sensor 135. The received signal may indicate an electrical potential of an area of tissue in contact with EMG sensor 135. In one embodiment, body area detection engine 400 may be configured to receive incoming signals communicated (e.g., pushed) by EMG sensor 135. In another embodiment, body area detection engine 400 may poll EMG sensor periodically, requesting a signal from EMG sensor 135. In any event, once the signal is received, body area detection engine 400 may execute operative position detection logic 410.

In one embodiment, body area detection engine 400 is a software module, and operative position detection logic 410 and body area detection logic 420 are embodied as one or more software functions. In this embodiment, the received signal may contain values representing the electrical potential of an area of skin in contact with EMG sensor 135. In this instance, the values contained in the signal are passed to operative position detection logic 410 and body area detection logic 420 as parameters of the function. In other embodiments, body area detection engine 400 and operative position detection logic 410 and body area detection logic 420 may be a combination of software and hardware components, such as an SoC or an ASIC.

In accordance with embodiments described herein, body area detection engine 400 and logic 410 and 420 may be hosted entirely by primary device 100, entirely by secondary device 150, or may be hosted partially on primary device 100 and partially on secondary device 150 (i.e., in any combination).

With further reference to FIG. 5, operative position detection logic 410 may start at step 505, where operative position detection logic 410 receives a value 507 that represents an electrical potential from some area of the user's body. In accordance with an embodiment, at step 510, received value 507 is evaluated with respect to a stored value 512. In one embodiment stored value 512 is stored in the memory/storage 310 of secondary BAN device 150. In one embodiment, stored value 512 is stored in the memory/storage 210 of primary device 100, which may be in communication with secondary device 150.

In accordance with an embodiment, stored value 512 may be a value representing a predefined electrical potential associated with a certain area of the body. For instance, predefined value 512 may be a value corresponding to an electrical potential expected at a user's wrist, finger, ear area, upper arm, lower arm, upper leg, lower leg, palm, foot, neck, eye socket, temple, etc. Further, in accordance with an embodiment, stored value 512 may be a range of values, where each value in the range is an acceptable expected value at a certain area of the user's body. For instance, stored value 512 may represent the range of −90 mV to −100 mV, or −75 mV to −80 mV, etc.

In one embodiment, the evaluation of step 510 includes a comparison of received value 507 and stored value 512. Received value 507 and stored value 512 may be compared to determine if the values match. In an embodiment where stored value 512 is a range of values, received value 507 may be determined to match stored value 512 if received value 507 falls in the range included in stored value 512.

In step 515, a determination is made as to whether secondary BAN device 150 is operatively positioned. This determination is based on the evaluation of step 510. For example, in an embodiment where the evaluation of step 510 includes comparing received value 507 and stored value 512, a positive determination may be made (i.e., secondary BAN device is operatively positioned) when the two values match. Conversely, a negative determination may be reached (i.e., secondary BAN device is not operatively positioned) if the two values do not match. In this way, body area detection logic may determine whether secondary BAN device is operatively positioned.

In the event that a negative determination is reached at step 515, body area detection logic 410 may proceed to step 520. At step 520, a negative determination value 522 is returned, and operative position detection logic 410 ends. On the other hand, in the event that a positive determination is made at step 515, body area logic proceeds to step 525, where a positive determination value 527 is returned, and operative position detection logic 410 also ends. In either scenario, body area detection engine 400 may continue to monitor for additional signals communicated from EMG sensor 135, and to pass any received values on to operative position detection logic 410 for evaluation.

It is contemplated that ranges of expected electrical potential for certain parts of a user's body may overlap. For instance, a range of expected electrical potential for a user's wrist may overlap with a range of expected electrical potential for a user's thigh. However, given the nature of secondary BAN enabled devices 150, and the specific areas secondary devices 150 are intended to be worn or placed on a user's body, such overlap in electrical potential will not prevent body area detection engine 400 from distinguishing when secondary device 150 is operatively positioned versus being in contact with another non-matching location of the user, such as in the user's hand.

As briefly discussed above, body area detection engine 400 may be hosted entirely on primary device 100, entirely on secondary device 150, or any combination thereof. In an embodiment where body area detection engine 400 is hosted on primary device 100, secondary device 150 may communicate the signal representing an electrical potential from EMG sensor 135 to body area detection engine 400 via the established BAN. In this case, primary device 100 may receive the signal over BAN contact 130, and body area detection engine 400, via operative position detection logic 410 may evaluate the signal. If a positive determination is returned by operative position detection logic 410, body area detection engine 400 may then notify control circuit 205 that secondary device 150 is in operative position. Upon receiving notification from body area detection engine 400 that secondary device 150 is in operative position, control circuit 205 may send, via the established BAN, functional controls to secondary device 150.

To illustrate, consider a scenario where the secondary device 150 is a BAN enabled door handle with a latch and functional instructions that cycle the latch and allow the door to open. The door handle may be considered operatively positioned when a user grasps the door handle with the palm of his or her hand. In such a scenario, the user may inadvertently touch the door handle with his or her elbow and a BAN would be established between primary device 100 and the door handle. The door handle may be configured to send primary device 100 a signal representing an electrical potential from EMG sensor 135 embedded in the door handle via the established BAN. Body area detection engine 400, located on primary device 100, may receive the signal transmitted across the established BAN. Operative position detection logic 410 may then evaluate the signal by comparing the received value 507 to a stored value 512 that represents the expected electrical potential value of the palm of the user's hand. However, received value 507 would represent the electrical potential of the user's elbow, and not the electrical potential of the user's palm. Therefore, received value 507 would not match stored value 512, and operative position detection logic 410 would return a negative determination. Thus, body area detection engine 400 may not notify control circuit 205 that secondary device 150 was operatively positioned. Control circuit 205 may be configured to not send functional instructions to device 150 unless a notification has been received that device 150 is operatively positioned, and, therefore, no functional instructions will be sent to secondary device 150. Because no functional instructions were sent, the door would not be unlatched due to the user's inadvertent touching of the BAN enabled door handle.

In an alternative embodiment where body area detection engine 400 is hosted on secondary device 150, body area detection engine 400 may receive the signal representing an electrical potential from EMG sensor 135, and operative position detection logic 410 may evaluate the signal by comparing the received value 507 to stored value 512, which represents the expected value of the palm of the user's hand. When a negative determination is made (e.g., the received value 507 did not match the stored value 512), body area detection engine 400 may simply not communicate a notification to primary device 100, or may notify primary device 100, via the established BAN connection, that secondary device 150 is not operatively positioned.

In either of the preceding two embodiments, should the user grasp the door handle with his or her palm (instead of inadvertently touching the door handle with an elbow), the evaluation of operative position detection logic 410 will indicate that secondary device 150 is operatively positioned (e.g., received value 507 matched stored value 512), and a positive determination value 527 will be returned from step 525. Body area detection module 400 will notify control circuit 205 that secondary device 150 is operatively positioned—either over the established BAN connection if body area detection engine 400 is hosted on secondary device 150, or via a local data bus if body area detection engine 400 is hosted on primary device 100. Upon receiving the notification from body area detection engine 400, control circuit 205 may be configured to send functional instructions to secondary device 150.

Another representative embodiment includes BAN enabled shoes, where the BAN enabled shoes are operatively positioned on the user's feet. Functional instructions for BAN enabled shoes may include instructions to start recording a user's steps when walking or running, or record a distance (via a global positioning sensor (GPS)) that the user has covered in the shoes.

Another representative embodiment includes a secondary BAN enabled headset 150 with a speaker and a primary BAN enabled media playing device. The BAN enabled headset 150 may be considered operatively positioned when placed on the user's head. Functional instructions for the BAN enabled headset 150 may include instructions to begin media playback. In this example, the BAN enabled media playing device may also contain an EMG sensor and operative position detection logic 410 may receive signals from the EMG sensors 135 of both the headset 150 and the media playing device. Control circuit 205 may be configured not to send functional instructions to headset 150 until both devices are determined to be operatively positioned and may cease when one or the other of the devices is no longer operatively positioned.

For example, media device may be considered operatively positioned when it is strapped to the use's upper arm. In this scenario, a positive determination that headset 150 is in operative position and a positive determination that the media player is in operative position must be made before control circuit 205 would send operative instructions from the media player to the headset. Further, in this scenario, functional instructions may merely include media playback, as mentioned above, or may be user-configured to include specific instructions. For instance, user defined functional instructions may include the playback of a certain playlist of music when both devices are in operative position.

Another representative embodiment includes a BAN enabled turnstile/payment device. The turnstile may prevent the user from freely entering an area that requires a fare—for instance, a subway or bus loading zone. The BAN enabled turnstile may be considered operatively positioned when the user touches a certain area of the turnstile with the user's palm, or perhaps the user's fingertips. The relevant functional instructions may include debiting an account of the user in order to pay the required fare, and releasing a lock on the turnstile in order to allow the turnstile to rotate and let the user proceed to the loading area.

In yet another representative embodiment, a piece of clothing may act as secondary BAN enabled device 150. For instance, a shirt or a hat would be considered operatively positioned when the shirt is worn by the user on the user's torso or hat is worn by the user on the user's head.

In accordance with embodiments described herein, body area detection engine 400 may continue to monitor for communication from EMG sensor 135, and EMG sensor 135 may continue to communicate signals to body area detection engine 400 while in the operative position. In such an embodiment, body area detection engine 400 may continue to pass received values to operative position detection logic 410 for evaluation. In one embodiment, if a negative determination value is returned to body area detection engine 400 (e.g., indicating that the device is no longer operatively positioned), body area detection engine 400 may be configured to stop sending functional instructions to secondary device 150, or alternatively, to terminate any functional instructions currently executing.

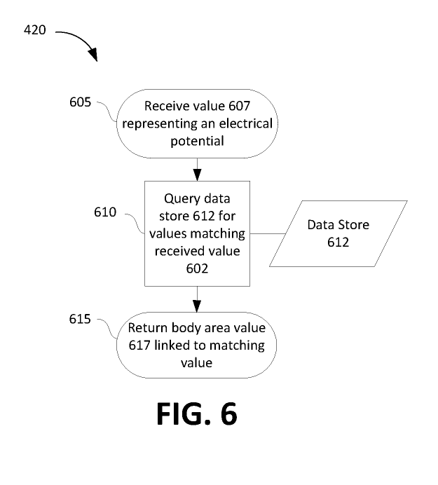

In still other embodiments, body area detection engine 400 may be configured to receive and evaluate more than one signal from the EMG sensor 135 of one or more secondary BAN enabled devices 150. With reference to FIG. 6, and in accordance with an embodiment, body area detection logic 420 may be configured to return a value representing the area of the body that is in contact with an EMG sensor 135.

Starting at step 605, body area detection logic may receive a value 607 representing an electrical potential. At step 610 body area detection logic 420 queries data store 612. Data store 612 may contain values that represent electrical potentials found at all different areas of the body, and each value that represents an electrical potential at a certain area of the body may be linked to another value which represents the area of the body at which the linked represented electrical potential may be found. In response to the query of step 610, data store 612 may return a value that matches received value 607 and the linked value representing the body area where such an electrical potential is found. In step 615 body area detection logic 420 may return body area value 617 (i.e., the linked value retrieved in the query of step 610).

Data store 612 may be a flat file, a relational database, key/value pairs, or any suitable data store. Data store 612 may be hosted on either primary device 100 or secondary device 150.

The predetermined values of data store 612 and predetermined stored value 512 may be populated using default values known to apply to a wide variety of potential users or through a user-performed learning procedure. In one embodiment of a user performed learning procedure for populating data store 612, a user specifies an area of his or her body to which the user is about to touch secondary device 150, and subsequently touches secondary device 150 to the indicated area. Values representing the specified area of the user's body and the electrical potential sensed by EMG sensor 135 of secondary device 150 are stored with the appropriate relationship. In another embodiment, a user may perform a learning procedure to record stored value 512. In this embodiment, the user may initialize the learning procedure, and subsequently operatively position secondary device 150. Once secondary device 150 is operatively positioned, a value representing the electrical potential sensed by the EMG sensor 135 of secondary device 150 is recorded and stored as stored value 512. In this way, the predetermined values of data store 612 and stored value 512 are personalized values, rather than generic values.

In other embodiments of the above learning procedures, the learning procedure is performed many times and average values of the sensed electrical potentials are recorded. In still another embodiment, a range of values is constructed through multiple performances of the learning procedure.

In accordance with an embodiment, each time a user touches a different area of his or her body with EMG sensor 135, a signal representing the electrical potential of that area of the user's body is sent to body area detection engine 400. Each of the several signals may be passed as a value 607 to body area detection logic 420. For each signal passed to body area detection logic 420, a body area value 617 is returned. Body area detection engine 400 may evaluate each returned body area value 617 in order to determine if a user has performed a predefined gesture by touching one or multiple parts of the user's body in a given sequence.

In one embodiment two secondary BAN enabled devices are configured to work in conjunction with a primary device 100. The two secondary devices 150 may be a BAN enabled ring 150 and a BAN enabled door handle with a lock. The relevant functional instructions may cause the door handle to cycle the lock, thereby locking or unlocking the door handle. Ring 150 may be operatively positioned when worn on the user's finger, and the door handle may be operatively positioned when grasped in the user's palm. Body area detection engine 400 may be configured to receive signals from the EMG sensors 135 of both the ring 150 and the door handle. Body area detection engine 400 may be configured to pass values received from the EMG sensors of both ring 150 and the door handle to operative position detection logic 410 to determine that the devices 150 are operatively positioned. Thus, when the user both wears the ring 150 on a finger and grasps the door handle with his or her palm, body area detection engine will notify control circuit 205 that both secondary devices 150 are operatively positioned, as described in detail above.

However, ring 150 may include an additional EMG sensor 135, for instance, on the outside of ring 150 (i.e., not in contact with the user's finger). In one embodiment, body area detection engine is configured to send signals received from the additional EMG sensor 135 not in contact with the user's finger to body area detection logic 420. Further, control circuit 205 may be configured to send functional instructions only when 1) a notification has been received indicating that both secondary devices 150 are in operative position (described in detail above), and 2) that a predefined gesture has been performed by the user.

In accordance with an embodiment, a user may touch ring 150 to one or several areas of the user's body. Each single touch, or each sequence of multiple touches may correspond to a predefined gesture. Each touch will result in a signal being sent from the additional EMG sensor 135 to body area detection engine 400, the signal representing the electrical potential of the area of the body touched by the user. Body area detection engine 400 may receive each signal and pass the value contained in each signal to body area detection logic 420. Body area detection logic 420 may, in turn, return a body area value 617 for each received value 607, as described in detail above. Body area detection engine may then evaluate each body area value 617, and the sequence in which the values 617 were received to determine if the body areas touched with ring 150 by the user match a predefine user gesture. If the gesture performed by the user matches a predefined gesture, then body area detection engine 400 may notify control circuit 205 that a predefined gesture has been performed by the user. At this point, both requirements needed by control circuit 205 will have been met—i.e., 1) a notification has been received indicating that both secondary devices 150 are in operative position, and 2) that a predefined gesture has been performed by the user. Thus, control circuit 205 will communicate functional instructions to the door handle and the lock will be cycled.

In another embodiment, secondary device 150 may contain other (alternate) types of sensors (e.g., accelerometers, capacitive touch sensors, etc., as described above). Gestures may be sensed by these other types of sensors, rather than by EMG sensor 135. Control circuit 205 may be configured to require both 1) a notification has been received indicating that secondary device 150 is operatively positioned (as described in detail above), and 2) that a predefined gesture has been performed by the user and sensed by the other alternate sensor. Control circuit 205 be configured with logic (not shown) to evaluate signals from alternate sensors to determine if the user has performed a predefined gesture.

Although certain embodiments have been shown and described, it is understood that equivalents and modifications falling within the scope of the appended claims will occur to others who are skilled in the art upon the reading and understanding of this specification.

1. A body area network system comprising:
a first body area network (BAN) enabled device comprising:
a control circuit that operatively controls the first BAN enabled device;
a BAN communication interface; and
an electromyography (EMG) sensor configured to sense an electrical potential of an area of tissue in contact with the EMG sensor;
a body area detection engine configured to:
receive a first signal communicated from the EMG sensor, wherein the first signal indicates the sensed electrical potential of an area of tissue in contact with the EMG sensor;
evaluate the indicated electrical potential with respect to a predetermined value;
determine, based on the evaluation, that the first device is operatively positioned with respect to a user;
cause the BAN communication interface to at least one of:
communicate a notification to a second BAN enabled device, the notification notifying the second device that the first device is operatively positioned; or
receive one or more functional instructions from the second BAN enabled device.

2. The system of claim 1, wherein the body area detection engine is part of the control circuit.

3. The system of claim 1, wherein the body area detection engine is part of a control circuit of the second BAN enabled device.

4. The system of claim 1, wherein the body area detection engine is hosted partially by the first BAN enabled device and hosted partially by the second BAN enabled device.

5. The system of claim 1, wherein the predetermined value is stored on the first BAN enabled device.

6. The system of claim 1, wherein the predetermined value is stored on the second BAN enabled device.

7. The system of any of claim 1, wherein the evaluation comprises comparing the indicated electrical potential and the predetermined value; and
wherein the evaluation indicates that the secondary BAN enabled device is operatively positioned on a user when the indicated electrical potential and the stored value match.

8. The system of any of claim 1, wherein the stored value represents a range of values, and wherein the indicated electrical potential matches the stored value when the indicated electrical potential is in the range of values.

9. The system of claim 1, wherein the EMG sensor communicates a second signal indicating a second sensed electrical potential of a second area of tissue contacted by the EMG sensor;
wherein the body area detection engine evaluates the second indicated electrical potential with respect to a second predetermined value; and
wherein the evaluation comprises determining, based on the second electrical potential indicated by the second signal and the second predetermined value, that a predetermined gesture has been performed by the user.

10. The system of claim 9, wherein the second signal is communicated by an other EMG sensor.

11. The system of claim 9, wherein the second signal is communicated by a third BAN enabled device.

12. The system of claim 1, wherein the first device contains an other input sensor and wherein the evaluation comprises further determining that a gesture has been performed by the user based on input from the other input sensor.

13. A method for determining that a first BAN enabled device is operatively positioned comprising:
sensing, via an EMG sensor integrated into the first BAN enabled device, an electrical potential of an area of tissue in contact with the EMG sensor;
communicating a first signal from the EMG sensor to a body area detection engine, wherein the signal indicates the sensed electrical potential of the tissue in contact with the EMG sensor;
evaluating, by the body area detection engine, the indicated electrical potential with respect to a predetermined value;
determining, based on the evaluating, that the first device is operatively positioned with respect to a user;
causing a BAN communication interface integrated into the first BAN enabled device to at least one of:
communicate a notification to a second BAN enabled device, the notification notifying the second device that the first device is operatively positioned; or
receive one or more functional instructions from the second BAN enabled device.

14. The method of claim 13, wherein the body area detection engine is hosted by the first BAN enabled device.

15. The method of claim 13, wherein the body area detection engine is hosted by the second BAN enabled device.

16. (canceled)

17. The method of claim 13, wherein the predetermined value is stored on the first BAN enabled device.

18. The method of claim 13, wherein the predetermined value is stored on the second BAN enabled device.

19. The method of claim 13, wherein the evaluating comprises comparing the indicated electrical potential and the predetermined value; and
wherein the evaluating further comprises indicating that the secondary BAN enabled device is operatively positioned on a user when the indicated electrical potential and the predetermined value match.

20. The method of claim 13, wherein the predetermined value represents a range of values, and wherein the indicated electrical potential matches the stored value when the indicated electrical potential matches any value in the range of values.

21. The method of claim 13, further comprising communicating a second signal indicating a second sensed electrical potential of a second area of tissue contacted by the EMG sensor to the body area detection module;
evaluating the second indicated electrical potential with respect to a second predetermined value;
determining, based on the electrical potential indicated by the second signal and the second predetermined value, that a predetermined gesture has been performed by the user.

22-30. (canceled)
